(19) United States
(12) Patent Application Publication
(10) Pub. No.: US 2023/0083955 A1
(43) Pub. Date: Mar. 16, 2023

(54) DOUBLE-GUSSETED LEAK-RESISTANT UNDERGARMENT WITH STRAPS

(71) Applicant: Amber LaTeal Robins, West Henrietta, NY (US)

(72) Inventor: Amber LaTeal Robins, West Henrietta, NY (US)

(21) Appl. No.: 17/931,120

(22) Filed: Sep. 10, 2022

Related U.S. Application Data
(60) Provisional application No. 63/261,239, filed on Sep. 15, 2021.

(51) Int. Cl. A61F 13/72; A61F 13/76

(52) U.S. Cl. CPC A61F 13/72; A61F 13/76

(57) ABSTRACT

The present invention is directed to an undergarment with a double gusset and leak-proof layer and loops or straps to hold absorbent pad of all sizes in place. The gussets provide multiple layers of wicking and absorbent fabrics for preventing leaks whether used with an absorbent pad or as back-up to tampon use.

### CROSS REFERENCE TO RELATED APPLICATIONS

This application claims priority to U.S. Provisional Application No. 63/261,239 entitled “Double-Gusseted Leak-Resistant Panty with Straps” and filed Sep. 15, 2021, which is hereby incorporated by reference.

### BACKGROUND OF THE INVENTION

### I. Field of the Invention

The present invention relates to an undergarment and, more particularly, to a double-gusseted leak-resistant undergarment with straps.

### II. Description of the Related Art

Many products have been developed for situations necessitating the use of an absorbent pad or undergarment, such as during a woman's menstrual period or when an individual experiences incontinence. These products, including feminine pads and adult diapers, attempt to protect clothing and help avoid embarrassment from fluid leaking through clothing. Unfortunately, many of these products tend to be excessively bulky and visible. When smaller products are available, such as a sanitary pad, the pad tends to move around so that blood or fluid may leak through the undergarment. Sanitary pads have also been developed with adhesive or other attachment devices; however, these attachment devices do not hold the pad as securely as advertised, can damage the undergarment when removing, or can leave adhesive on the undergarment when removed. Other products attempt to hold the pad in place, but do not accommodate all pad sizes.

Accordingly, there is a need for an undergarment that prevents leaks and securely holds an absorbent pad of any size in place. The undergarment is also useable as an absorbent and leak-proof garment when using tampons or when incontinence is an issue.

### BRIEF SUMMARY OF THE INVENTION

The present invention includes both leak-proof technology as well as a double gusset. It both helps keep an absorbent pad in place and prevents leaks whether used with an absorbent pad or as a back-up to tampon use. It also accommodates absorbent pads and sanitary napkins of all sizes.

Broadly, an embodiment of the present invention provides an undergarment comprising a double gusset, a leak-resistant layer, and straps. The panty comprises a double gusset along the crotch area between the legs. The double gusset provides an area to which the user may locate sanitary napkin attachment extensions, known commonly as “wings.” A top leak-resistant gusset along the inner crotch of the undergarment provides three (3) layers to help prevent menstrual blood or other fluid from leaking through the undergarment. Two (2) straps, one at the front and another at the back of the top gusset, cross the undergarment crotch laterally to help keep the napkin or pad in place.

The invention may be manufactured by sewing together various cloth materials that have excellent wicking and absorbency characteristics and a material that is leak resistant.

The inventive undergarment may be used to hold an absorbent pad or sanitary napkin in place or as a backup for menstrual bleeding or other fluid that may leak when using a tampon.

It is therefore an object of the present invention to provide an undergarment with multiple gussets of materials providing leak resistance and absorbency to protect against leaks beyond the undergarment.

It is also an object of the present invention to provide an undergarment with a variety of fabrics to accommodate the various leak resistance and absorbency requirements.

It is a further object of the present invention to provide an undergarment that provides secure placement and use of an absorbent pad or sanitary napkin.

It is a further object of the present invention to provide additional locations to attach preexisting attachment extensions of sanitary napkins, such as wings.

These and other features and advantages will be set forth in the description that follows, and in part will be apparent from the description, or may be learned by practice of the invention.

The objectives and other advantages of the present invention will be realized and attained by the structure particularly pointed out in the written description and claims hereof, as well as the appended drawings.

It is to be understood that both the forgoing general description, and the following detailed description are exemplary and explanatory, and are intended to provide further explanation of the invention as claimed.

### BRIEF DESCRIPTION OF THE DRAWINGS

FIG. 1a is a front elevation view of an undergarment according to an embodiment of the present invention;

FIG. 1b is a rear elevation view of an undergarment according to an embodiment of the present invention;

FIG. 2 is a top plan view showing an undergarment separated at the sides and laid flat according to a further embodiment of the present invention;

FIG. 3 is a close-up perspective view of the gusset configuration according to an embodiment of the present invention;

FIG. 4 is a detailed cross-section view of the gussets according to an embodiment of the present invention; and

FIG. 5 is an additional top plan view of an undergarment according to an embodiment of the present invention.

### DETAILED DESCRIPTION OF THE INVENTION

The following detailed description provides various preferred embodiments of carrying out the invention. The description is not to be taken in a limiting sense but is made merely for the purpose of illustrating the general principles of the invention.

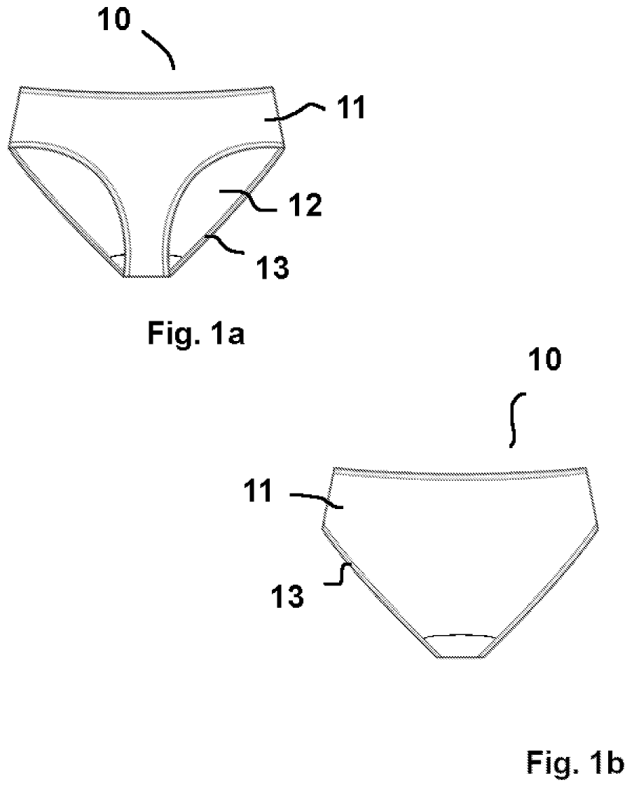

Turning now to the drawings in more detail, FIG. 1a provides a front view of undergarment 10 and FIG. 1b shows a rear view of the undergarment 10, according to various embodiments of the present invention. FIG. 1a shows the undergarment body layer 11, the garment inner lining 12, and the elastic leg seals 13. FIG. 1b shows the undergarment body layer 11 and elastic leg seals 13. According to various embodiments of the present invention the front and rear views of undergarment 10 outwardly appear as a typical undergarment.

According to one embodiment of the present invention, body layer 11 and inner lining layer 12 are made from fabric made of 95% bamboo and 5% spandex. It will be apparent to those skilled in the art that a variety of fabrics may be used to fabricate the body layer 11 and inner lining layer 12 of undergarment 10 to provide the appropriate comfort and functionality of undergarment 10 according to the scope and spirit of the present invention.

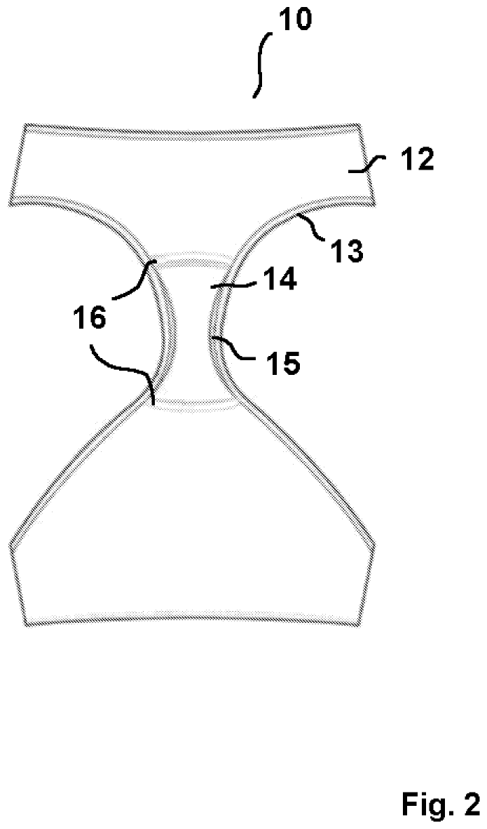

FIG. 2 shows undergarment 10 separated on the sides and laid flat to display the details of the inner construction. Various embodiments of the present invention provide for the sides to attach with buttons, hook and loop, or are simply sewn together. According to the embodiment shown in FIG. 2, undergarment 10 includes the inner lining 12, the elastic binding 13 in the leg openings, an inner leak-proof gusset 14, an outer folded gusset 15, and elastic loops 16 across gussets 14 and 15.

According to the embodiment shown in FIG. 2, outer gusset 15 is sewn laterally along the leg openings in the crotch area of undergarment 10. The outer gusset 15 is typically constructed of a layer of lining fabric, a leak-proof layer, and a breathable fabric layer sewn to the body fabric 11 of undergarment 10 along with the elastic binding 13 of the leg openings. The leak-proof layer and the breathable fabric layer are typically fused together to provide support and strength for the leak-proof layer; however, various configurations will be apparent based on available fabrics. Outer gusset 15 provides a second leak-proof layer to ensure that any excess fluid does not leak past the outer gusset 15 and into the user's clothing.

Inner gusset 14 is typically constructed of a layer of lining fabric, a moisture wicking layer, an absorbent layer, a leak-proof layer, and a breathable fabric. The leak-proof layer and the breathable fabric layer are typically fused together to provide support and strength for the leak-proof layer; however, various configurations will be apparent based on available fabrics. The moisture wicking fabric of inner gusset 14 draws moisture away from the user toward the absorbent layer, which holds any fluids reaching inner gusset 14. Inner gusset 14 is placed over gusset 15 and attached to the undergarment 10 at the top and bottom of gusset 14 in which the stitching of gusset 14 is perpendicular to the stitching of gusset 15. Fastening inner gusset 14 along the top and bottom and outer gusset 15 along the sides leaves the sides of gusset 14 unattached providing a gap along the leg openings of undergarment 10 between gusset 14 and gusset 15.

The opening between inner gusset 14 and outer gusset 15 provides a location for sanitary napkin attachment extensions, commonly known as wings, to be placed to further secure the sanitary napkin within undergarment 10. Additionally, elastic loops 16 cross the undergarment laterally to securely hold an absorbent pad or sanitary napkin in place above gusset 14. Elastic loops 16 hold a pad or napkin whether they have wings or not.

According to various embodiments of the present invention, inner gusset 14 and outer gusset 15 may be limited to the crotch area of undergarment 10 or extended to any point up to the waist band of undergarment 10.

Elastic binding 13 along the leg openings provides a further level of protection against leaks between the undergarment 10 and the user's legs.

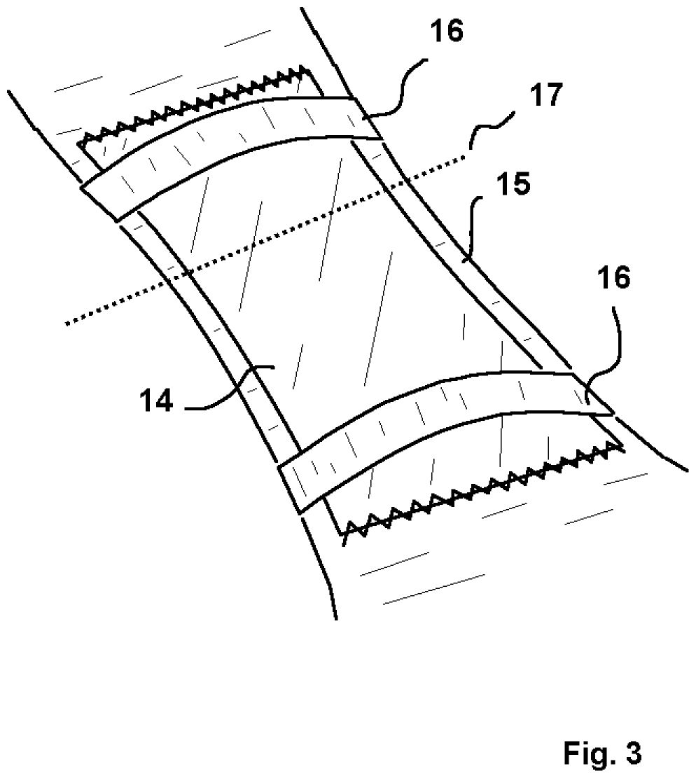

FIG. 3 provides further detail to the arrangement of the inner gusset 14, the outer gusset 15, and elastic strap 16 configuration. As shown in FIG. 3, outer gusset 14 is sewn to the undergarment 10 at the top and bottom of the gusset 14. Straps 16 are attached laterally across inner gusset 14 and outer gusset 15.

Elastic straps 16 are used to hold an absorbent pad or sanitary napkin between gusset 20 and the user. The elastic nature of the straps 16 allow for a variety of sizes of pads to be used in the undergarment 10. In further embodiments, elastic straps 16 may be elastic mesh or other fabrics providing the ability to hold pads in place.

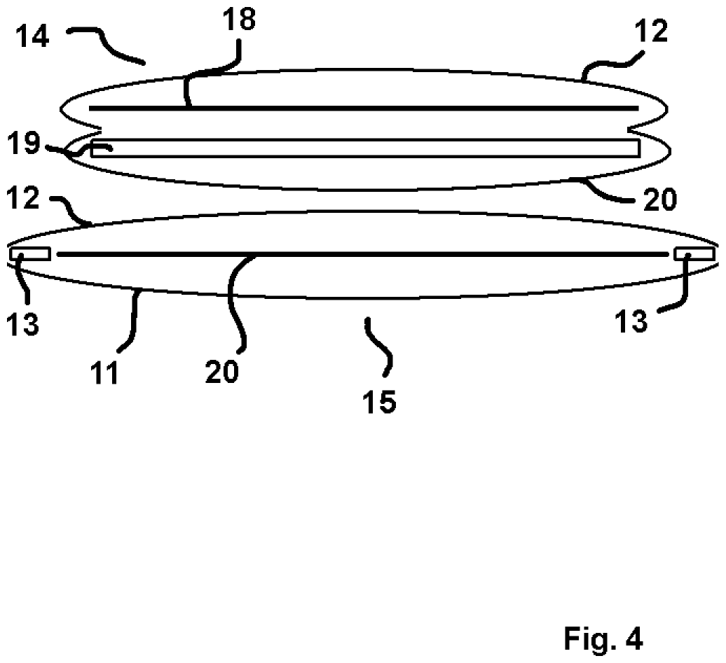

FIG. 3 also includes a bi-section line 17 to be used in FIG. 4 to show the individual layers.

FIG. 4 shows the layers of inner gusset 14 and outer gusset 15 at the bisection line 17 shown in FIG. 3. As described above, inner gusset 14 includes a layer of lining fabric 12, a moisture wicking layer 18, an absorbent layer 19, and a leak-proof and breathable fabric layer 20. The leak-proof and the breathable fabric layer 20 are typically fused together to provide support and strength for the leak-proof lining, such as a thermoplastic polyurethane membrane (hereinafter “TPU”); however, various options and configurations as a single layer or multiple layers will be apparent to one skilled in the art based on available fabrics and technology. The moisture wicking fabric layer 18 draws moisture away from the user toward the absorbent layer 19. As shown in FIG. 4, inner gusset 14 is placed above gusset 15.

According to an embodiment of the present invention, the lining fabric layer 12 is made of 95% bamboo and 5% spandex; wicking layer 18 is made of a fabric of 56% bamboo, 24% cotton, and 20% polyester; absorbent layer 19 is made of 95% cotton and 5% spandex; and a leak-proof layer 20 using a breathable TPU membrane bonded to a breathable fabric.

Further shown in FIG. 4, is outer gusset 15. As described above, outer gusset 15 includes a lining layer 12, a leak-proof layer 20, and the outer body layer 11. The layers of the outer gusset 15 are attached together along the elastic leg seals 13. According to an embodiment of the present invention, the lining layer 12 is a fabric made of 95% bamboo and 5% spandex; the leak-proof layer is constructed of a TPU layer bonded to a breathable fabric; and the outer layer 11 is a fabric of 95% bamboo and 5% spandex.

FIG. 4 further shows the space between inner gusset 14 and outer gusset 15. The two-gusset construction provides a space for the wings of a pad to be contained within undergarment 10 and help the pad stay in place. The two gussets also provide two leak-proof layers to ensure any fluid escaping from the inner gusset 14 is blocked from reaching the user's clothing by the leak-proof layer 20 of the outer gusset 15.

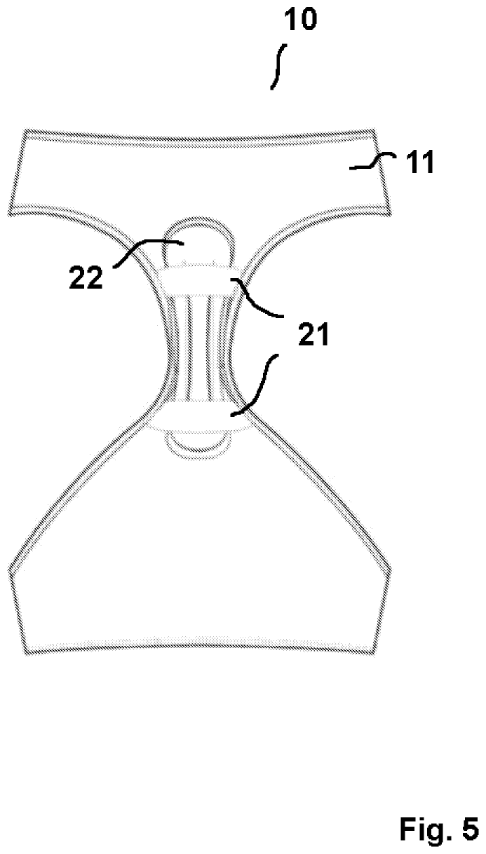

FIG. 5 provides another view of undergarment 10 according to a further embodiment of the present invention. As shown in FIG. 5, undergarment 10 is separated on the sides and laid flat to display the details of the inner construction. The undergarment 10 of FIG. 5 incorporates mesh straps 21 for holding an absorbent pad or sanitary napkin 22 in place within undergarment 10. A further embodiment of the present invention includes mesh straps 21 made of an elastic mesh to further enhance their ability to hold the pad in place.

It will be clear to one skilled in the art that a variety of fabrics may be used for the various parts of undergarment 10 and that fabrics will be developed with features that will continue to enhance the use and structure of undergarment 10.

It should be understood that the foregoing relates to exemplary embodiments of the present invention. Further additions or alterations may be made to the undergarment without departing from the spirit and scope of the present disclosure. Furthermore, those skilled in the art will understand that a number of variations, including those described, may be made to the fabrics, materials, other elements, and the processes for making the undergarment, all without departing from the scope of the invention, which is described above and defined by the appended claims.

I claim:

1. A double-gusseted leak-resistant undergarment comprising:
a base undergarment;
a first gusset attached to the crotch area of the base undergarment;
a second gusset attached above the first gusset; and
two or more straps attached laterally across the second gusset for securing an absorbent pad.

2. A double-gusseted leak-resistant undergarment according to claim 1 wherein the first gusset is attached to the undergarment along the leg openings.

3. A double-gusseted leak-resistant undergarment according to claim 1 wherein the second gusset is attached to the undergarment along at the top and bottom of the second gusset creating a gap between the first gusset and the second gusset.

4. A double-gusseted leak-resistant undergarment according to claim 1 wherein the first gusset further comprises a wicking and absorbent material.

5. A double-gusseted leak-resistant undergarment according to claim 1 wherein the second gusset further comprises a leak-proof material.

6. A double-gusseted leak-resistant undergarment according to claim 1 wherein the two or more straps further comprise elastic straps.

7. A double-gusseted leak-resistant undergarment according to claim 1 wherein the two or more straps further comprise mesh straps.

8. A double-gusseted leak-resistant undergarment according to claim 1 further comprising elastic binding in said leg openings.
